(19) United States
(12) Patent Application Publication
(10) Pub. No.: US 2019/0192330 A1
(43) Pub. Date: Jun. 27, 2019

(54) Single Pull Lumbar Support Brace

(71) Applicants: Trevor J. Theriot, Newport Beach, CA (US); John N. Lasso, Laguna Hills, CA (US)

(72) Inventors: Trevor J. Theriot, Newport Beach, CA (US); John N. Lasso, Laguna Hills, CA (US)

(21) Appl. No.: 15/851,514

(22) Filed: Dec. 21, 2017

(51) Int. Cl. A61F 5/02; A61F 5/32

(52) U.S. Cl. CPC A61F 5/028; A61F 5/32

(57) ABSTRACT

A single pull lumbar support brace that comprises of a central molded plate. The central molded plate supports a three-piece pulley system. The three-piece pulley system comprises of a left buckle, a right buckle, a linear rigid gear holder and a single cord that attaches to a pull handle. The linear rigid gear holder separates the left and right buckles and prevents the cord from tangling when tension is applied or released from the pulley system. A left and a right belt attach to the left and the right buckles of the central molded plate, thereby creating a single pull lumbar support brace that is tangle proof.

### BACKGROUND

The present invention is directed to a single pull lumbar support brace.

Lumbar support braces are presently used to treat spine disorders, to heal from current conditions affecting the lumbar area of the spine, or to prevent injuries when conducting physical activities such as heavy lifting.

After a lower back injury is sustained by an individual, a lumbar support brace is used to control pain, reduce the chance of further injuries, allow healing to take place, or to compensate muscle weakness.¹ ¹Available at http://www.umm.edu/programs/spine/health/guides/braces, BRACES, University of Maryland Medical Center, Dec. 2, 2017.

Braces have been around at least as early as 2000 BC,² and the present invention is a single pull lumbar support brace that allows a user to adjust the tension using a pulley system that can be manipulated by a two-finger ergonomic pull handle. ²Id.

Lumbar supports are described in the following patents: U.S. Pat. Nos. 6,213,968, 8,663,141, 7,001,348, 9,393,149, 8,956,315, 8,142,377, and D725,786. The above referenced patents are used to provide a lumbar support, yet they do not accomplish the support in the manner of the present invention.

The present invention improves the manner of supporting the lumbar area of the spine by allowing the user to adjust the tension of the lumbar support brace by pulling a single ergonomic pull handle that is attached to cord that is connected to an improved pulley system that defines non-tangling system.

For the foregoing reasons there is a need for a single pull lumbar support brace that will allow a user to adjust the tension created within the lumbar support brace via a pulley system that is tangle proof.

### SUMMARY

The present invention describes a single pull lumbar support brace that can be adjusted by pulling a pull handle with two fingers. The pull handle is connected to a pulley system that is mounted on the support brace. The pulley system defines a non-tangling tension mechanism.

The present invention comprises of a single pull lumbar support brace that comprises of: a molded plate that has a left, a right, an upper, a lower side, a front, and a rear side, the upper rear side; a left buckle, the left buckle defines a left linear aperture and has three linear gears that are parallel to the left linear aperture, the left buckle is attached to the left rear side of the molded plate so that the left linear aperture is adjacent to the left side of the molded plate; a right buckle, the right buckle defines a right linear aperture and has a first set and a second set of two linear gears that are parallel to the right linear aperture, the right buckle is attached to the right rear side of the molded plate so that the right linear aperture is adjacent to the right side of the molded plate; a rigid linear gear holder, the rigid linear gear holder defines six linear gears that evenly spaced on the rigid linear gear holder, the rigid linear gear holder is attached to the rear side of the molded plate so that it is centered between the left and right buckle and so that the six linear gears run from the upper to the lower side of the molded plate; a cord having two ends, the cord is looped through the gears of the left buckle, the rigid linear gear holder and the right buckle, and the ends of the cord are attached to pull handle; a left belt that has a left, a right, a rear and a front side, the right side of the left belt attaches to the left buckle at the left linear aperture, the left belt defines a linear hook and loop receiver on the rear side of the belt that runs from the left side of the left belt, for at least 6 inches, towards the right side the left belt, and a hook and loop end receiver that covers the left rear side of the left belt; and a right belt that has a left, a right, a rear and a front side, the left side of the right belt attaches to the right buckle at the right linear aperture, the right belt defines a linear hook and loop receiver on the rear side of the right belt that runs from the right side of the right belt, for at least 6 inches, towards the left side the right belt, and a hook and loop end connector that covers the right front side of the right belt.

The present invention is used by placing the single pull lumbar support brace around the lumbar area of a user. Then, connecting the hook and loop connector and receiver of the left and right belt of the brace to establish a first tension. Then pulling the pull handle of the brace with two fingers to establish a second tension along the central horizontal linear axis of the lumbar support brace. The tension is established when the left and right buckles are pulled inward by the cord when the pull handle is pulled outward from the left buckle. Upon obtaining the desired tension, then the pull handle is secured on to either the left or the right belt linear hook and loop receiver.

An object of the present invention is to provide a single pull lumbar support brace that uses pulley system to establish a certain tension.

Another object of the present invention is to provide a single lumbar support brace that uses a pulley system to establish tension that incorporates a tangle proof mechanism in its pulley system.

Yet another object of the present invention is to provide a single lumbar support brace that has an ergonomic two-finger pull handle.

### DRAWINGS

These and other features, aspects, and advantages of the present invention will become better understood with regards to the following description, appended claims, and drawings where:

FIG. 1 is a perspective view of the present invention on a user;

FIG. 2 is a rear view of the present invention;

FIG. 3 is a front view of the present invention;

FIG. 4 is rear view of the present invention that shows how adjustable pads would be placed on the belts of the brace;

FIG. 5 is a rear view of the ergonomic two-fingered pull handle;

FIG. 6 is a rear view of the pulley system of the present invention in a first position; and

FIG. 7 is a rear view of the pulley system of the present invention is a second position.

### DESCRIPTION

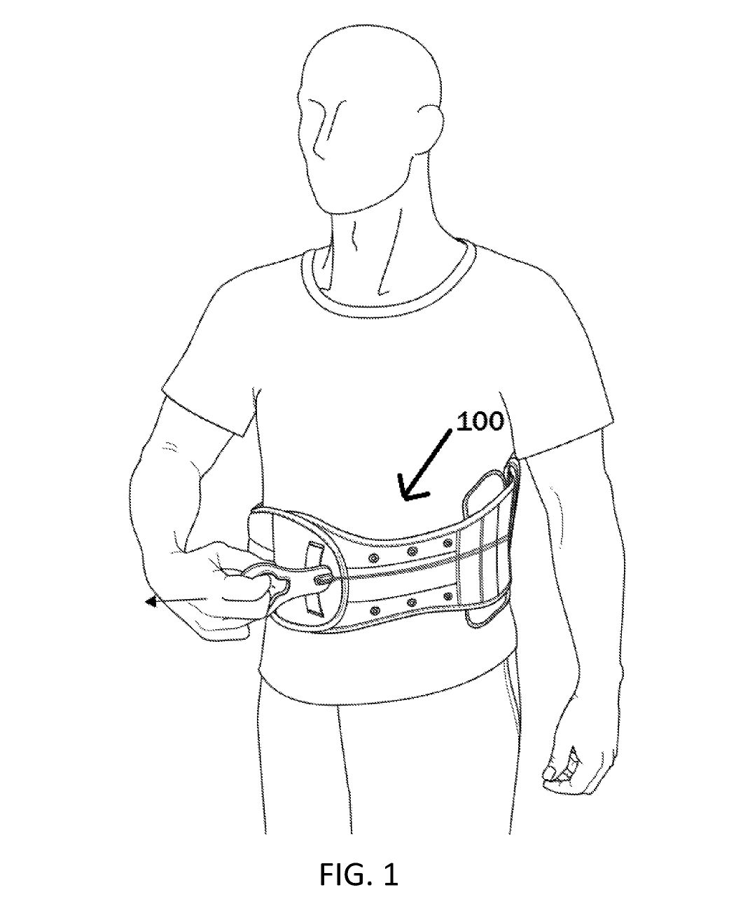

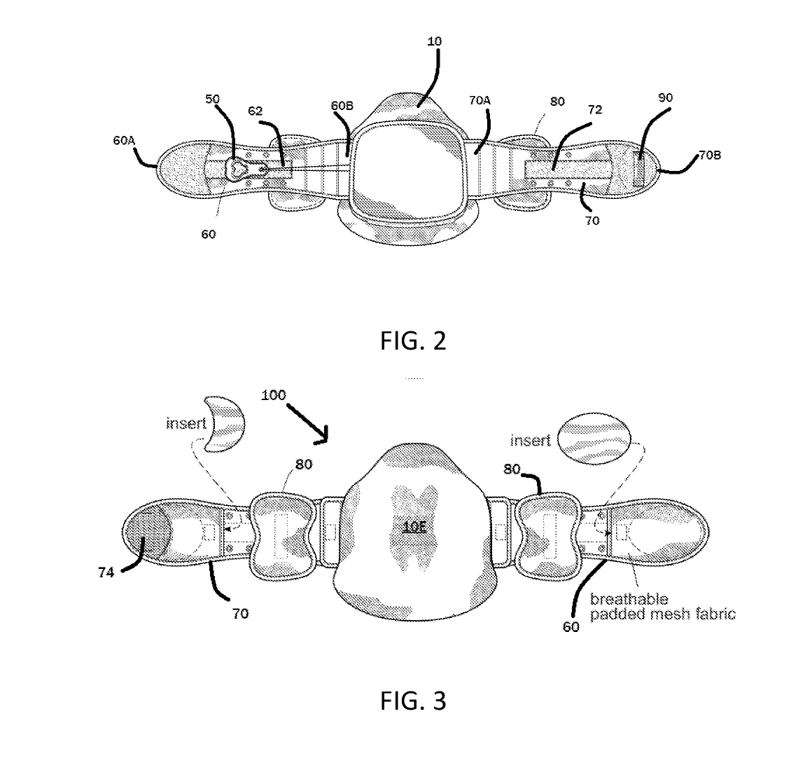

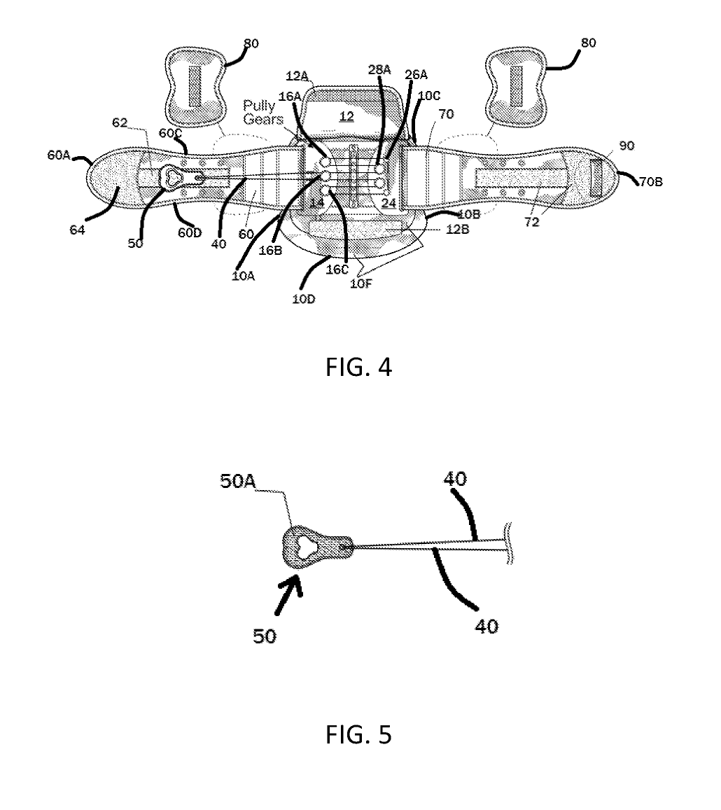

As seen in FIGS. 1-6, the present invention is a single pull lumbar support brace 100, the brace 100 comprises of a molded plate 10 that has a left 10A, a right 10B, an upper 10C, a lower side 10D, a front 10E, and a rear side 10F, the upper rear side of the plate 10C defines a flap 12 that has a hook and loop connector 12A, and the lower rear side 100 defines a hook and loop receiver 12b; a left buckle 14, the left buckle 14 defines a left linear aperture 14A and has three linear gears 16A, 16B, 16C that are parallel to the left linear aperture 14A, the three linear gears 16A, 16B, 16C are an upper 16A a middle 16B and a lower gear 16C, the left buckle 14 is attached to the left rear side of the molded plate 10 so that the left linear aperture 14A is adjacent to the left side of the molded plate 10A; a right buckle 24, the right buckle 24 defines a right linear aperture 24B and has a first set 26A,26B and a second set of two linear gears 28A,28B that are parallel to the right linear aperture 24B, each set of the two linear gears has an upper gear 26A,28A and a lower gear 26B,28B, the first set of linear gears 26A,26B are closer to the right linear aperture 24A and are positioned outward, when measured from the upper and lower side of the molded plate 10, of the second set of linear gears 26A,26B, the right buckle 24 is attached to the right rear side of the molded plate 10 so that the right linear aperture 24A is adjacent to the right side of the molded plate 10B; a rigid linear gear holder 30, the rigid linear gear holder 30 defines six linear gears that evenly spaced on the rigid linear gear holder 30, the rigid linear gear holder 30 is attached to the rear side of the molded plate 10F so that it is centered between the left 14 and right buckle 24 and so that the six linear gears run from the upper 10C to the lower side 10D of the molded plate 10 and the gears are defined from the lower side to the upper side as gear a 32A, gear b 32B, gear c 32C, gear d 32D, gear e 32E, and gear f 32F; a cord 40 having two ends, the cord 40 is looped through the gears of the left buckle 14, the rigid linear gear holder 30 and the right buckle 24, and the ends of the cord 40 are attached to pull handle 50; a left belt 60 that has a left 60A, a right 60B, a rear 60C and a front side 60D, the right side 60B of the left belt 60 attaches to the left buckle 14 at the left linear aperture 140, the left belt 60 defines a linear hook and loop receiver 62 on the rear side of the belt that runs from the left side of the left belt 60A, for at least 6 inches, towards the right side the left belt 60B, and a hook and loop end receiver 64 that covers the left rear side of the left belt 60; and a right belt 70 that has a left 70A, a right 70B, a rear 70D and a front side 70C, the left side of the right belt 70A attaches to the right buckle 24 at the right linear aperture 24A, the right belt 70 defines a linear hook and loop receiver 72 on the rear side of the right belt that runs from the right side of the right belt 70B, for at least 6 inches, towards the left side the right belt 70A, and a hook and loop end connector 74 that covers the right 70B front side 70C of the right belt 70.

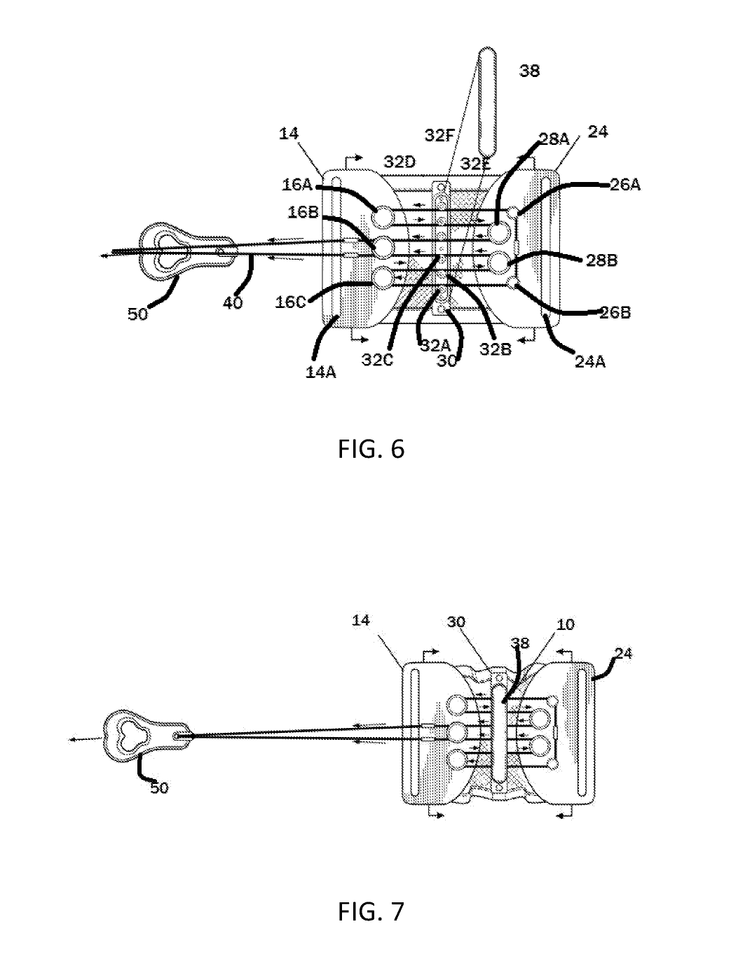

As seen in FIGS. 6-7, in an embodiment, the cord loops 40 around the left buckle 14, the rigid linear gear holder 30 and the right buckle 24 as follows: it first is passes through a lower side of middle gear 16B of the left buckle 14, then it passes through an upper side of the gear c 32C of the rigid linear gear holder 30, next it loops around the lower gear 28B of second set of linear gears of the right buckle 24, then it passes through an upper side of gear b 32B of the rigid linear gear holder 30, next loops around the lower gear 16C of the left buckle 14, then it passes through an upper side of gear a 32A of the rigid linear gear holder 30, next it passes through a lower side of the lower gear 26b of the first set of linear gears of the right buckle 24, then it passes through a right side of the upper gear 26A of the first set of linear gears of the right buckle 24; next it passes through a lower side of gear f 32F of the rigid linear gear holder 30, then it loops around the upper gear 16A of the left buckle 14, next it passes through a lower side of gear e 32E of the rigid linear gear holder 30, then it loops around the upper gear 28A of the second set of linear gears of the right buckle 24, next it passes through a lower side of gear d 32D of the rigid linear gear holder 30, and lastly it passes through an upper side of the middle gear 16B of the left buckle 14.

In another embodiment of the present invention, the pull handle 50 is a two-finger ergonomic pull handle, and the pull handle 50 has a hook and loop connector 50A on one side.

In yet another embodiment of the present invention the front sides of the left and right belt define a hook and loop pad receiver (not shown in figures) and the lumbar support brace comprises of a pair of adjustable pads 80 that define a hook and loop connector (not shown in figures). The hook and loop connectors of each adjustable pad 80 attaches to each hook and loop pad receiver.

In still another embodiment of the present invention, the rear right belt defines a loop handle 90 that receives the pull handle.

In yet still another embodiment, the support brace comprises of a cover 38 that attaches to the linear rigid gear holder.

An advantage of the present invention is that it provides a single pull lumbar support brace that uses a pulley system to establish a certain tension.

Another advantage of the present invention is that it provides a single lumbar support brace that uses a tangle proof mechanism in its pulley system.

Yet another advantage of the present invention is that it provides a single lumbar support brace that has an ergonomic two-finger pull handle.

While the inventor's above description contains many specificities, these should not be construed as limitations on the scope, but rather as an exemplification of several preferred embodiments thereof. Many other variations are possible. Accordingly, the scope should be determined not by the embodiments illustrated, but by the appended claims and their legal equivalents.

What is claimed is:

1. A single pull lumbar support brace, the brace comprises:
A molded plate that has a left, a right, an upper, a lower side, a front, and a rear side, the upper rear side of the plate defines a flap that has a hook and loop connector, and the lower rear side defines a hook and loop receiver;
A left buckle, the left buckle defines a left linear aperture and has three linear gears that are parallel to the left linear aperture, the three linear gears are an upper a middle and a lower gear, the left buckle is attached to the left rear side of the molded plate so that the left linear aperture is adjacent to the left side of the molded plate;
A right buckle, the right buckle defines a right linear aperture and has a first set and a second set of two linear gears that are parallel to the right linear aperture, each set of the two linear gears has an upper gear and a lower gear, the first set of linear gears are closer to the right linear aperture and are positioned outward, when measured from the upper and lower side of the molded plate, of the second set of linear gears, the right buckle is attached to the right rear side of the molded plate so that the right linear aperture is adjacent to the right side of the molded plate;
A rigid linear gear holder, the rigid linear gear holder defines six linear gears that evenly spaced on the rigid linear gear holder, the rigid linear gear holder is attached to the rear side of the molded plate so that it is centered between the left and right buckle and so that the six linear gears run from the upper to the lower side of the molded plate and the gears are defined from the lower side to the upper side as gear a, gear b, gear c, gear d, gear e, and gear f;
A cord having two ends, the cord is looped through the gears of the left buckle, the rigid linear gear holder and the right buckle, and the ends of the cord are attached to pull handle;
A left belt that has a left, a right, a rear and a front side, the right side of the left belt attaches to the left buckle at the left linear aperture, the left belt defines a linear hook and loop receiver on the rear side of the belt that runs from the right side of the left belt, for at least 6 inches, towards the right side the left belt, and a hook and loop end receiver that covers the left rear side of the left belt; and
A right belt that has a left, a right, a rear and a front side, the left side of the right belt attaches to the right buckle at the right linear aperture, the right belt defines a linear hook and loop receiver on the rear side of the right belt that runs from the right side of the right belt, for at least 6 inches, towards the left side the right belt, and a hook and loop end connector that covers the right front side of the right belt.

2. The single pull lumbar support brace of claim 1, wherein the cord loops around the left buckle, the rigid linear gear holder and the right buckle as follows: it first is passes through a lower side of middle gear of the left buckle, then it passes through an upper side of the gear c of the rigid linear gear holder, next it loops around the lower gear of second set of linear gears of the right buckle, then it passes through an upper side of gear b of the rigid linear gear holder, next loops around the lower gear of the left buckle, then it passes through an upper side of gear a of the rigid linear gear holder, next passes through a lower side of the lower gear of the first set of linear gears of the right buckle, then it passes through a right side of the upper gear of the first set of linear gears of the right buckle; next it passes through a lower side of gear f of the rigid linear gear holder, then it loops around the upper gear of the left buckle, next it passes through a lower side of gear e of the rigid linear gear holder, then it loops around the upper gear of the second set of linear gears of the right buckle, next it passes through a lower side of gear d of the rigid linear gear holder, and lastly it passes through an upper side of the middle gear of the left buckle.

3. The single pull lumbar support brace of claim 2, wherein the pull handle is a two-finger ergonomic pull handle, and the pull handle has a hook and loop connector on one side.

4. The single pull lumbar support brace of claim 3, wherein the front side of the left and right belts define a hook and loop pad receiver and the lumbar support brace comprises of a pair of adjustable pads that define a hook and loop connector, the hook and loop connectors of each adjustable pad attaches to each hook and loop pad receiver.

5. The single pull lumbar support brace of claim 4, wherein the rear right belt defines a loop handle that receives the pull handle.

6. The single pull lumbar support brace of claim 5, the support brace comprises a cover that attaches to the linear rigid gear holder.
